(19) United States
(12) Patent Application Publication
(10) Pub. No.: US 2024/0307892 A1
(43) Pub. Date: Sep. 19, 2024

(54) CONTINUOUS CENTRIFUGAL ISOLATING SYSTEM AND METHODS OF USE THEREOF

(71) Applicant: The Regents of the University of Colorado, a body corporate, Denver, CO (US)

(72) Inventors: Kang H. Song, Superior, CO (US); Mark A. Boden, Boulder, CO (US); Jong Shin, Aurora, CO (US)

(21) Appl. No.: 18/271,993

(22) PCT Filed: Jan. 11, 2022
(86) PCT No.: PCT/US22/11986

§ 371 Date: Jul. 12, 2023

Related U.S. Application Data
(60) Provisional application No. 63/136,352, filed on Jan. 12, 2021.

(51) Int. Cl. B04B 11/02; A61B 8/08; B04B 5/10; B04B 9/00

(52) U.S. Cl. CPC B04B 11/02; B04B 5/10; B04B 9/00; A61B 8/481

(57) ABSTRACT

The inventive technology described herein includes a novel mechanical apparatus and system which allows centrifugation of small micron sized particles. The invention allows for continuous separation of ultrasound contrast agents (microbubbles) from lipid solution via centrifugation assisted by a motor. The system allows for mass isolation and collection of microbubbles. The invention also allows for the dispensation of lipid solution. In one preferred embodiment, dilute microbubble solution is introduced to the system and subjected to a centrifugal force such that the particles migrate towards the center and collectively rises for extraction, while the lipid solution dispenses and is replace with an equal volume of dilute solution. In one preferred embodiment, the device includes a rotating column or cylinder driven by a belt drive assembly, with the column being supported by annular bearings on both ends. The bearings allow rotation of the outer cylinder independent of stationary input cylinder and output components.

### CROSS-REFERENCE TO RELATED APPLICATIONS

This International PCT application claims the benefit of and priority to U.S. Provisional Application No. 63/136,352 filed Jan. 12, 2021. The specification, claim and drawings of which are all incorporated herein by reference in their entirety.

### TECHNICAL FIELD

The invention relates to novel systems, methods and apparatus for the continuous separation of dilute buoyant particles, and in particular ultrasound contrast agents (microbubbles) from lipid solution via centrifugation.

### BACKGROUND

Interest in the use of targeted microbubbles for ultrasound molecular imaging (USMI) has been growing in recent years as a safe and efficacious means of diagnosing tumor angiogenesis and assessing response to therapy. Of particular interest are cloaked microbubbles, which improve specificity by concealing a coupled ligand from blood components until they reach the target vasculature, where the ligand can be transiently revealed for firm receptor-binding by ultrasound acoustic radiation force pulses. Microbubbles are approved in over seventy countries for use in routine ultrasound diagnosis of a wide variety of medical abnormalities of the heart, liver, gastro-intestinal tract, kidneys and other organ systems. At the forefront of this technology are targeted microbubbles, which are being developed for USMI of specific vascular phenotypes, such as inflammation and angiogenesis. Human clinical trials of USMI using microbubbles targeted to biomarkers of tumor angiogenesis were recently reported for noninvasive diagnosis of ovarian, breast and prostate cancers.

Typically, microbubbles are generated from the mechanical agitation (i.e., sonicator, impeller) of a stock solution (in one embodiment, lipids dispersed in water at approximately 2 mg/mL). This mechanical agitation generates a low concentration of buoyant microbubbles, generally containing a mixture of microbubbles and surfactant (i.e., lipid, PEG40 Stearate) solution, generally referred to herein as a dilute microbubble suspension. However, the microbubbles must be further concentrated for reasons of packaging, handling, dosing, and drug-delivery efficiency. Currently, microbubbles are concentrated in a batch-wise fashion, relying heavily on conventional rotor-and-bucket centrifuge systems. Compared to the continuous isolation of microbubbles described herein, conventional batch-wise processing lengthens the time and cost necessary to generate concentrated microbubbles by requiring additional steps, such as the ejection of infranatant, frequent input from human operators to operate the centrifuge, and the requirement of additional extraneous components, such as syringes that are required for batch-production of microbubbles.

As can be seen, there exists a need for a cost-effective and efficient microbubble concentration and isolation system that addresses the concerns outlined above.

### SUMMARY OF THE INVENTION

One aspect of the current inventive technology includes a novel separating system of small micron sized particles that allows for their continuous separation from solution via centrifugation. Another aspect of the current inventive technology includes a novel separating system of small micron sized particles that allows for separation of ultrasound contrast agents (microbubbles) from lipid solution via centrifugation. Another aspect of the current inventive technology includes a continuous centrifugal isolation system that allows for the continuous concentration and extraction of buoyant micron sized particles, such as ultrasound contrast agents (microbubbles) from lipid solution via centrifugation.

Another aspect of the current inventive technology includes a continuous centrifugal isolation system having a centrifugal column configured to hold a continuous flow of dilute buoyant particles in a suspension. Rotation of the centrifugal body, such as a column or cylinder, exerts a centrifugal force on the dilute buoyant particles causing them to migrate to the center of the rotating body forming a region of concentrated buoyant particles. Conversely, the centrifugal body exerts a centrifugal force on the components of the solution, such as excess lipids or surfactants, causing them to migrate outward forming a region of excess solution, such as an excess lipid solution.

Another aspect of the current inventive technology includes methods and systems for gravity assisted extraction of the concentrated buoyant particles centrally positioned in a centrifugal column. In this preferred aspect, the differential buoyancy of the concentrated buoyant particles, compared to the excess lipid solution, induces the upward migration of the concentrated buoyant particles within the centrifugal column facilitating extraction.

Another aspect of the current inventive technology includes methods and systems for generating a fluid flow along the central axis of the centrifugal column. In this preferred aspect, the centrifugal column may be supported by one or more annular bearings on both ends. The bearings allow rotation of the outer cylinder independent of stationary input cylinder and output cylinders. In this specific aspect, a quantity of dilute micron sized buoyant particles in a suspension may be inserted into the centrifugal column through the stationary input cylinder forming a fluid flow along the central axis of the centrifugal column causing the upward migration of the concentrated buoyant particles within the centrifugal column.

In another aspect, a continuous centrifugal isolation system may further include a stationary output cylinder positioned at the top of the centrifugal column and include at least one extraction port aligned with the central axis of the rotating cylinder, such that the upwardly migrating concentrated buoyant particles can be directed to the extraction port for removal. An extraction pump may further be coupled with the stationary output cylinder and in fluid communication with the centrifugal column so as to generate an upward fluid flow further inducing the upward migration of the concentrated buoyant particles within the centrifugal column.

Another aspect of the invention may include systems and methods for the continuous operation of a centrifugal isolation system through the coordinated addition and dispensation of dilute solution, concentrated particle suspension, and/or excess lipid or surfactant solution, respectively. In this preferred aspect, a quantity of dilute particle solution is introduced to the centrifugal column and subjected to a centrifugal force causing the buoyant particles to migrate towards the center forming a region of concentrated particle solution which further migrates upward through the column for extraction. The quantities of dilute particle solution introduced to the column and concentrated particle solution that is extracted may be coordinated so as to maintain an approximate equilibrium of the volume in the column. In another aspect, a quantity of excess lipid solution may also be removed from the centrifugal column and recycled back through the system for further particle concentration or placed in an excess lipid solution reservoir. The quantities of dilute particle solution introduced to the column and concentrated particle solution and/or excess lipid solution that is extracted may be coordinated so as to maintain an approximate equilibrium of the volume in the column. This input and dispensation cycle may be repeated allowing the continuous concentration and extraction of a concentrated particle solution.

Another aspect of the current inventive technology includes a continuous centrifugal isolation system having a centrifugal column configured to hold a continuous flow of dilute microbubble solution. Rotation of the centrifugal column exerts a centrifugal force on the dilute microbubble solution causing the dilute microbubbles to migrate to the center of the rotating cylinder forming a region of concentrated microbubbles, also generally referred to herein as a concentrated microbubble solution. Conversely, the centrifugal column exerts a centrifugal force on the components of the solution, such as excess lipids or surfactants, forming, causing them to migrate outward forming a region of an excess lipid solution.

Another aspect of the current inventive technology includes methods and systems for gravity assisted extraction of a concentrated microbubble solution centrally positioned in the centrifugal column. In this preferred aspect, the differential buoyancy of the concentrated buoyant microbubbles, compared to the excess lipid solution, induces the upward migration of the concentrated buoyant microbubbles within the centrifugal column facilitating extraction.

Another aspect of the current inventive technology includes methods and systems for generating a fluid flow along the central axis of the centrifugal column. In this preferred aspect, the centrifugal column may be supported by one or more annular bearings on both ends. The bearings allow rotation of the column independent of stationary input cylinder and output cylinder components. In this specific aspect, a quantity of dilute microbubble solution may be inserted into the centrifugal column through the stationary input cylinder forming a fluid flow along the central axis of the centrifugal column causing the induces the upward migration of the concentrated buoyant microbubbles within the centrifugal column.

In another aspect, a continuous centrifugal isolation system may further include a stationary output cylinder positioned at the top of the centrifugal column and include at least one extraction port aligned with the central axis of the rotating cylinder, such that the upwardly migrating concentrated buoyant microbubbles can be directed to the extraction port for removal. An extraction pump may further be coupled with the stationary output cylinder and in fluid communication with the centrifugal column so as to generate an upward fluid flow further inducing the upward migration of the concentrated buoyant microbubbles within the centrifugal column.

Another aspect of the invention may include systems and methods for the continuous operation of a centrifugal isolation system through the coordinated addition and dispensation of dilute microbubble suspension, concentrated microbubble suspension, and/or excess lipid solution, respectively. In this preferred aspect, a quantity of dilute microbubble solution is introduced to the centrifugal column and subjected to a centrifugal force causing the microbubbles to migrate towards the center forming a region of concentrated microbubble solution which further migrates upward through the column for extraction. The quantities of dilute microbubble solution or suspension introduced to the column and concentrated particle solution or suspension that is extracted may be coordinated so as to maintain an approximate equilibrium of the volume in the column. In another aspect, a quantity of excess lipid solution may also be removed from the centrifugal column and recycled back through the system for further microbubble concentration or placed in an excess lipid solution reservoir. The quantities of dilute microbubble suspension introduced to the column and concentrated microbubble solution and/or excess lipid solution that is extracted may be coordinated so as to maintain an approximate equilibrium of the volume in the column. This input and dispensation cycle may be repeated allowing the continuous concentration and extraction of a concentrated microbubble suspension from the column.

Another aspect of the invention may include a continuous centrifugal isolation system that is configured to isolate microbubbles in a sterile environment. Another aspect of the invention may include a continuous centrifugal isolation system that is configured to feed to a microbubble dehydration system. Another aspect of the invention may include a continuous centrifugal isolation system that is configured to generate at least 50 mL of concentrated microbubble suspension per minute.

Other aspects and features of the present invention will become apparent to those ordinarily skilled in the art upon review of the following descriptions of specific embodiments of the invention in conjunction with the accompanying figures.

### BRIEF DESCRIPTION OF THE FIGURES

The above and other aspects, features, and advantages of the present disclosure will be better understood from the following detailed descriptions taken in conjunction with the accompanying figures, all of which are given by way of illustration only, and are not limiting the presently disclosed embodiments, in which:

FIG. 1: shows a method of isolating and extracting a concentrated microbubble suspension from a dilute microbubble solution introduced to a centrifugal column in one embodiment thereof;

FIG. 2: shows a side view of a continuous centrifugal isolation system in one embodiment thereof;

FIG. 3: shows a perspective view of a continuous centrifugal isolation system having a centrifugal column rotatably coupled with a belt drive in one embodiment thereof;

FIG. 4: shows a side view of a continuous centrifugal isolation system having a centrifugal column rotatably coupled with a belt drive in one embodiment thereof;

FIG. 5: shows a close up view of a centrifugal column and a stationary input cylinder and a stationary output cylinder independently positioned at the bottom and top of the column respectively in one embodiment thereof;

FIG. 6: shows a close up view of a centrifugal column coupled with an annular bearing and coupled with a cylinder support in one embodiment thereof;

FIG. 7: shows a close up view of a belt drive system configured to rotate the centrifugal column in one embodiment thereof;

FIG. 8: shows a top view of a belt drive system configured to rotate the centrifugal column in one embodiment thereof;

FIG. 9: shows two views of a stationary output cylinder having an internally positioned extraction port in one embodiment thereof;

FIG. 10: shows two views of a stationary input cylinder having an internally positioned input port, and two drain ports positioned laterally on the body of the cylinder in one embodiment thereof; and

FIG. 11: shows an exemplary scaffold having a top plate, middle plate, and bottom plate in one embodiment thereof.

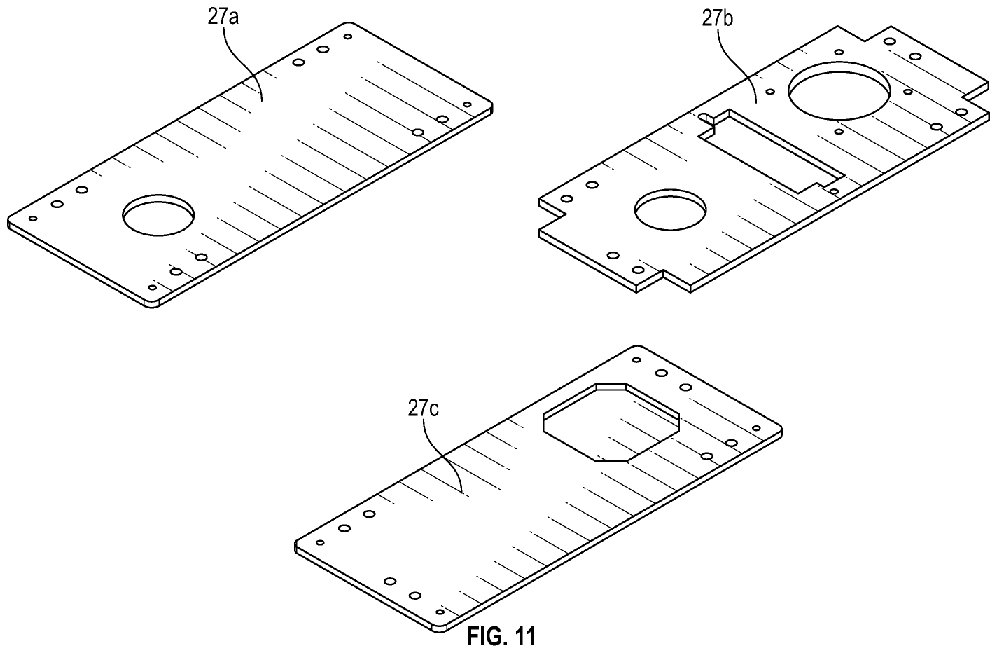

### DETAILED DESCRIPTION OF THE INVENTION

The present invention includes a variety of aspects, which may be combined in different ways. The following descriptions are provided to list elements and describe some of the embodiments of the present invention. These elements are listed with initial embodiments; however, it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

As noted above, microbubbles may be generated through mechanical agitation of a lipid solution, such as sonication or being introduced to an impeller or other similar device or process. The formed microbubbles must be concentrated prior to their use in various diagnostic and therapeutic applications. The inventive technology provides for a continuous centrifugal isolation system (1) configured to concentrate and extract buoyant particles, such as preferably microbubbles, for therapeutic and diagnostic uses, and the like.

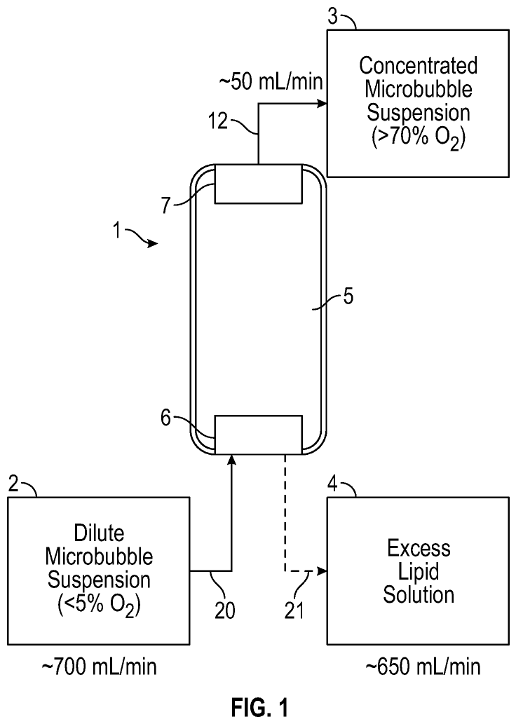

Generally referring to FIG. 1, a continuous centrifugal isolation system (1) of the invention includes one or more centrifugal columns (5) or rotational bodies or cylinders, (the terms being generally interchangeable herein) that are configured to continuously: i) receive a quantity of a dilute microbubble suspension (2); ii) apply a centrifugal force causing the microbubble structures in the dilute microbubble suspension (2) to migrate to the center of the centrifugal column (5) forming a concentrated microbubble suspension (3) region and an excess lipid solution (4) in the outer region of the centrifugal column (5); and iii) extract the concentrated microbubble suspension (3) from the system; and iv) optionally drain a quantity excess lipid solution (4) from the centrifugal column. In certain preferred embodiments, the quantity of a dilute microbubble suspension (2) may be approximately the same as the concentrated microbubble suspension (3) and may be input and extracted at approximately the same rate so as to maintain an approximate equilibria within the centrifugal column (5) allowing for the continuous concentration and extraction of microbubbles from the system.

Notably, as used herein, a “dilute microbubble suspension” generally refers to a volume of a low-concentration microbubble suspension, such as a suspension of unconcentrated microbubble structures. In one embodiment, a dilute microbubble suspension (2) may include a suspension of microbubbles having not undergone any form of microbubble concentration. In one preferred embodiment, a dilute microbubble suspension (2) may be prepared through one or more methods known in the art and have 5% or less encapsulated oxygen by volume. In another preferred embodiment, a dilute microbubble suspension (2) may be prepared through one or more methods known in the art and have a concentration of microbubbles of 10⁸ MB/mL or less.

As used herein, a “concentrated microbubble suspension” generally refers to a volume of high-concentration microbubble suspension, such as a suspension of concentrated microbubble structures. In one preferred embodiment, a concentrated microbubble suspension (3) may be prepared by undergoing a microbubble concentration process, and preferably the continuous centrifugal isolation process of the invention. In one embodiment, a concentrated microbubble suspension (3) may have 70% or greater encapsulated oxygen by volume. As detailed below, the invention may generate a thin column of concentrated microbubbles along the central axis of a rotating centrifugal column (5), which may also generally be referred to as a concentrated microbubble suspension (3). In another preferred embodiment, a concentrated microbubble suspension (2) may have a concentration of microbubbles of 10⁹ MB/mL or more.

As used herein, an excess “excess lipid solution” generally refers to a volume of the leftover portion of a dilute microbubble suspension (2) after undergoing a microbubble concentration process, and preferably the continuous centrifugal isolation process of the invention. In one preferred embodiment, an excess lipid solution (4) may contain a higher concentration of lipids and/or surfactants than a dilute microbubble suspension (2), and a lower concentration of microbubbles. In another embodiment, an excess lipid solution (4) may contain substantially no microbubbles.

In one preferred embodiment, the centrifugal force generated by the rotation of the centrifugal column (5) may be between 10-300 or more RCF/Gs, or any rotational speed sufficient to cause the migration of microbubbles to the center of the rotating body.

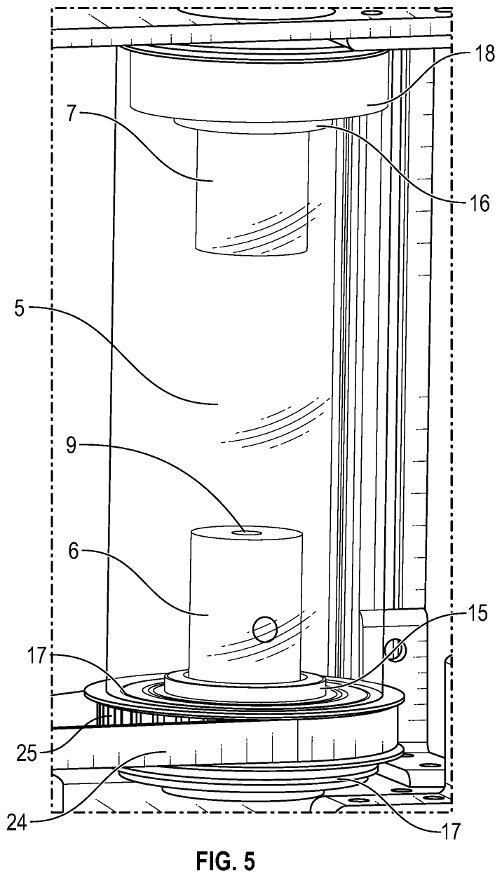

The invention may further include an apparatus for the continuous centrifugal isolation of dilute buoyant particles. Generally referring to FIGS. 1-5, the system may include a centrifugal column (5) configured to hold a continuous flow of dilute buoyant particles, for example in the form of a quantity of dilute microbubble suspension (2). In this preferred embodiment, the centrifugal column (5) may include one or more rotational cylinders supported by one or more bearing, and preferably annular bearings (17) positioned at the terminal ends of the centrifugal column (5). In this configuration, the centrifugal column (5) may be independently rotatable in response to the application of a rotational force as discussed below.

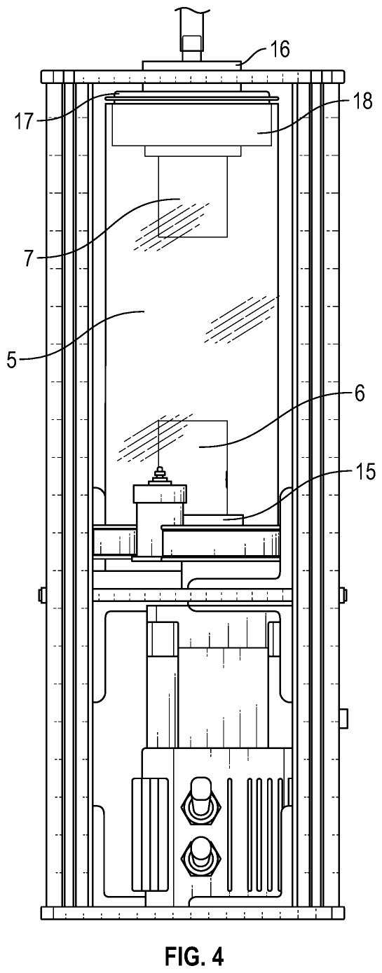

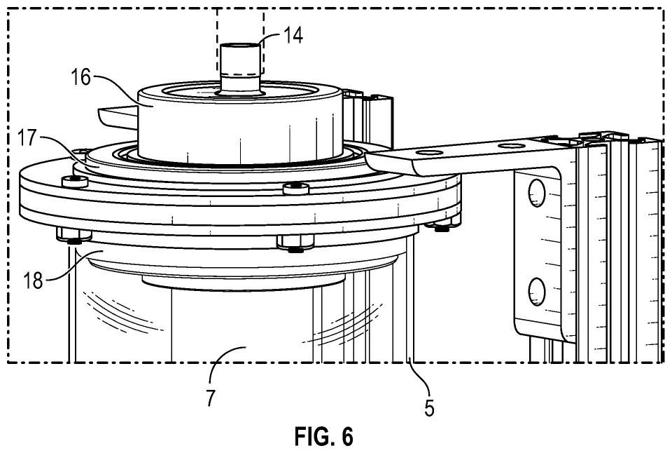

As shown in FIGS. 4-6, the centrifugal column (5) of the invention may include one or more cylinder supports (18) coupled to the terminal ends of the centrifugal column (5). In this configuration, the upper and lower cylinder supports (18) may form a water-tight seal between the outside environment and the internal area of the centrifugal column (5). The upper and lower terminal cylinder supports (18) may further be coupled with upper and lower input and output supports (15, 16), respectively. The input and output supports (15, 16) support the centrifugal column and may further be coupled with, and/or responsive to upper and lower annular bearing (17) further allowing the system of the invention to include a water-tight and independently rotatable centrifugal column (5), configured to concentrate dilute buoyant particles, such as microbubbles.

While in one embodiment of the invention the centrifugal column (5) is shown to have an initial volume capacity of approximately 700 mL, this initial capacity is exemplary only, and not limiting on the size, shape or dimension of a centrifugal column (5) that may be used with the invention.

The continuous centrifugal isolation system (1) may further be configured to generate a centrifugal force causing the dilute buoyant particles, and preferably microbubbles, to migrate to the center of the centrifugal column (5) forming a region having a concentrated microbubble suspension (3) positioned around the rotational or central axis of the centrifugal column (5).

Generally referring to FIGS. 1-6, a quantity of dilute microbubble suspension (2) may be generated and introduced to a centrifugal column (5) through a stationary input cylinder (6). In this preferred embodiment, a stationary input cylinder (6) may be positioned at the bottom terminal end of the centrifugal column (5) and may be coupled with a scaffold (27) or other stationary component. In this configuration, the stationary input cylinder (6) may be in fluid communication with the centrifugal column (5), however it is uncoupled and not responsive to the rotational movement of the column (5) thereby being maintained in a stationary position even as the centrifugal column (5) is rotated.

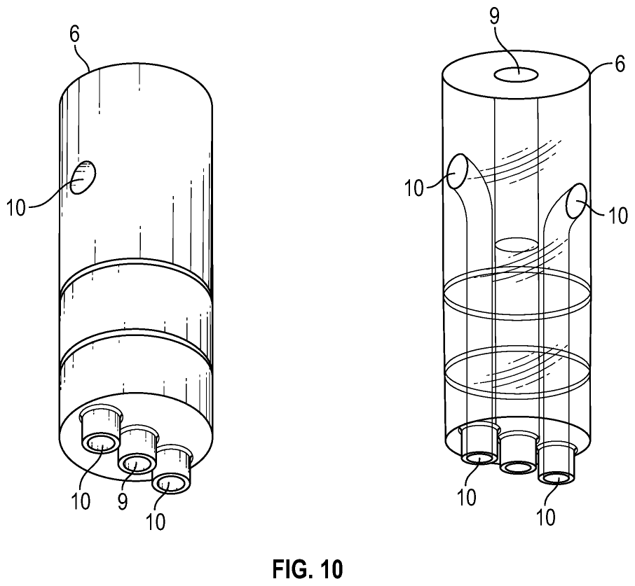

As shown in FIG. 10, a stationary input cylinder (6) may include one or more input ports (9) configured to receive and introduce a quantity of dilute microbubble suspension (2) into the internal area of the centrifugal column (5). The rate of introduction of dilute microbubble suspension (2) may be controlled by an input pump (20) in fluid communication with a reservoir or other quantity of dilute microbubble suspension (2), preferably through an input pipe (11b).

A quantity of a dilute microbubble suspension (2) is introduced to the internal area of the centrifugal column (5) through the input port (9). The rotational movement of the centrifugal column (5) generates a centrifugal force directed outward from the column that is applied to the dilute microbubble suspension (2). The buoyancy of the dilute buoyancy particles, in this embodiment microbubbles, causes them to migrate opposite the direction of the centrifugal force, concentrating them at the center of the centrifugal column (5). This process causes a thin region of concentrated microbubbles to form along the central axis of the centrifugal column (5). Conversely, in response to the centrifugal force generated by the rotation of the column (5), the less buoyant lipids and/or surfactants of the dilute microbubble suspension (2) are forced outward forming a region of excess lipid solution (4) positioned along the outer radius or portion of the centrifugal column (5).

Generally referring to FIGS. 1-6, a quantity of concentrated microbubble suspension (3) may be generated within the centrifugal column (5) and extracted through a stationary output cylinder (7). In this preferred embodiment, a stationary output cylinder (7) may be positioned at the top terminal end of the centrifugal column (5) and may be coupled with a scaffold (27) or other stationary component. In this configuration, the stationary output cylinder (7) may be in fluid communication with the centrifugal column (5), however, similar to the stationary input cylinder (6), it is uncoupled and not responsive to the rotational movement of the column (5) thereby being maintained in a stationary position even as the centrifugal column (5) is rotated.

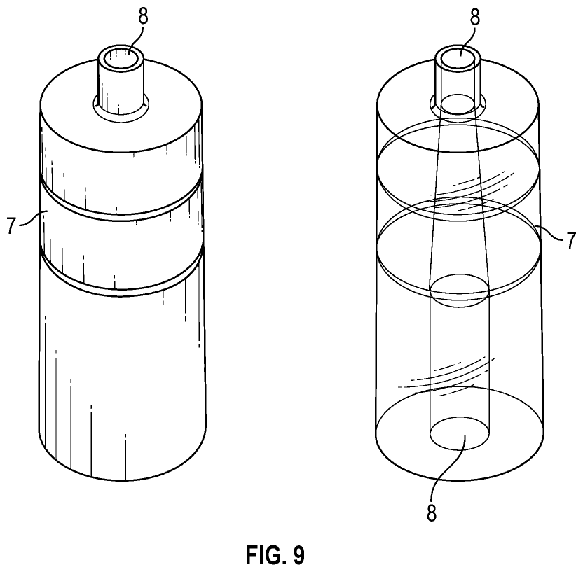

As shown in FIG. 9, a stationary output cylinder (7) may include one or more extraction ports (9) configured to extract a quantity of concentrated microbubble suspension (3) generated along the central axis of the centrifugal column (5). As noted above, the microbubble structures within the concentrated microbubble suspension (3) region of the centrifugal column (5) may have a greater buoyancy than the surrounding solution. In this embodiment, the buoyancy of the microbubbles is sufficient to overcome the natural gravitational force applied to the system, causing the microbubble structures within the concentrated microbubble suspension (3) region of the centrifugal column (5) to migrate upward. In a preferred embodiment, the extraction port (8) of the stationary output cylinder (7) may be aligned with the central axis of the centrifugal column (5), such that the upwardly migrating column of microbubbles are extracted by passing through the extraction port (8).

The extracted concentrated microbubble suspension (3) may be transferred to a reservoir (13). In one preferred embodiment, a stationary output cylinder (7) may be coupled with an extraction pipe (11a) by a pipe coupler (14), that may further include one or more valves (19) that may be manually or automatically operated in response to a signal generated by a sensor (30) transmitted to a digital device (29) having a processing system configured to effect one or more executable applications in response to a signal from the one or more sensors (30).

The continuous centrifugal isolation system (1) may further be configured to generate an upward fluid flow through the centrifugal column (5), and in particular along the central axis of the column to generate the upward migration of concentrated microbubbles. As noted above, the stationary input cylinder (6) and stationary output cylinder (7) may be placed at the opposing ends of the centrifugal column (5). In a preferred embodiment, the stationary input cylinder (6) and stationary output cylinder (7) may be positioned such that the input port (9) and the extraction port (8) may be aligned. In this configuration, the input of dilute microbubble suspension (2) through the input port (9) may cause an upward fluid force pushing the microbubble structures within the concentrated microbubble suspension (3) region of the centrifugal column (5) upward towards the extraction port (8) of the stationary output cylinder (7). Also, in this configuration, the extraction of concentrated microbubble suspension (3) may draw from the central region of the centrifugal column (5), generating an additional fluid force pulling the microbubble structures within the concentrated microbubble suspension (3) region of the centrifugal column (5) upward towards the extraction port (8). Notably, the rate of fluid flow may be adjusted through increasing or decreasing the action of the input pump, or extraction pump (20, 12) respectively.

The input pump (20) may further be responsive to one or more sensors (30) that may detect one or more parameters, such as rate of input or exhaustion of liquid from the centrifugal column (5), quantity of microbubbles in the input or exhaustion suspensions. The sensor (30) of the invention may generate a signal that may be transmitted to a digital device (29) having a processing system configured to effect one or more executable applications in response to the signal from one or more sensors (30) that may affect the rate of fluid input into the centrifugal column, or its rotational speed.

The rate of extraction of concentrated microbubble suspension (3) may be controlled by an extraction pump (12) in fluid communication with a reservoir (13) or other quantity of concentrated microbubble suspension (3). The extraction pump (12) may further be responsive to one or more sensors (30) that may detect one or more parameters, such as rate of input or exhaustion of liquid from the centrifugal column (5), quantity of microbubbles in the input or exhaustion suspensions, or quantity of concentrated microbubble suspension in a reservoir (13). The sensor (30) of the invention may generate a signal that may be transmitted to a digital device (29) having a processing system configured to effect one or more executable applications in response to the signal from one or more sensors (30) that may affect the rate or fluid extraction from the centrifugal column.

As the concentrated microbubble suspension (3) is extracted, and new dilute microbubble suspension (2) is introduced from the centrifugal column (5), the increasing fraction of excess lipid and/or surfactant solution can be exhausted to prevent the re-dilution of the concentrated microbubble suspension (3) being extracted the system. This diminishment of excess lipid and/or surfactant solution, generally referred to herein as excess lipid solution (4) may be achieved through a system and apparatus to drain excess lipid solution (4).

In one embodiment, the continuous centrifugal isolation system (1) of the invention may further include a system to equalize the volume within the centrifugal column and to further allow continuous isolation and extraction of microbubbles. As shown in FIG. 1, in one embodiment, the invention may include a drain configured to exhaust a quantity of fluid, and preferably excess lipid solution (4) from the internal area of the centrifugal column (5). In one embodiment a drain may be configured to exhaust excess lipid solution (4) from the outer radius of the centrifugal column (5) where the excess lipid solution (4) accumulates in response to the centrifugal force generated by the rotating centrifugal column (5).

In one preferred embodiment shown in the figures, a cylinder input may include one or more drain ports (10), which in this embodiment are shown on the stationary input cylinder (6), although they can alternatively be placed on the stationary output cylinder (7), or elsewhere in the system that is in fluid communication with the outer radius of the centrifugal column (5). As shown in FIG. 10, one, or a plurality of drain ports (10) may be positioned laterally along on body of the stationary input cylinder (6). The drain port(s) (10) may be in fluid communication with a drain pump (21), for example through a drain pipe (11c) allowing a quantity of excess lipid solution (4) to be extracted from the internal area of the centrifugal column (5). Further, by being placed laterally along on body of the stationary input cylinder (6), the drain ports (10) draw from the excess lipid solution (4) deposited in the outer radius or portion by the rotational force of the centrifugal column (5). Further, in this configuration, the drain port(s) (10) of the invention may be positioned outside the radius, and in this instance below, the column of concentrated microbubble suspension (3) generated by the system as described above.

In additional embodiments, the drain port (10) of the invention may allow exhaustion of excess lipid solution (4) from the centrifugal column (5) without the aid of a pump. In this configuration, the rate of exhaustion of excess lipid solution (4) may be controlled by a valve (12), or alternatively by the aperture size of the drain port (10), which may optionally be coupled with a convoluted pathway to further control the rate of exhaustion. In this latter embodiment, gravity may allow exhaustion of excess lipid solution (4) at a controlled rate.

As noted above, the rate of exhaustion of excess lipid solution (4) may be controlled by a drain pump (21) in fluid communication with a reservoir (13) or other appropriate receptacle. The drain pump (21) may further be responsive to one or more sensors (30) that may detect one or more parameters, such as rate of input or exhaustion of liquid from the centrifugal column (5), quantity of microbubbles in the input or exhaustion suspensions, or quantity of concentrated microbubble suspension in a reservoir (13). The sensor (30) of the invention may generate a signal that may be transmitted to a digital device (29) having a processing system configured to effect one or more executable applications in response to the signal from one or more sensors (30) that may affect the rate or excess lipid solution (4) extraction from the centrifugal column.

Notably, the continuous centrifugal isolation system (1) of the invention may further be configured to maintain an equalization of intake volume (input port) vs. exhaust volume (extraction port and, drain port). For example, as shown in FIG. 1, in one preferred embodiment the system may include an approximate rate of ˜200 mL/min for the introduction of a dilute microbubble solution, an approximate rate of ˜50 mL/min for the extraction of a concentrated microbubble suspension (3), as well as an approximate rate of ˜650 mL/min for the exhaustion rate of an excess lipid solution (4). Such rates and volumes are exemplary only, and not limiting on the various rates of the input, extraction of exhaustion of a volume of liquid from the system. In addition, equalization of intake volume may be accomplished through the action of an input port and extraction port only in certain embodiments.

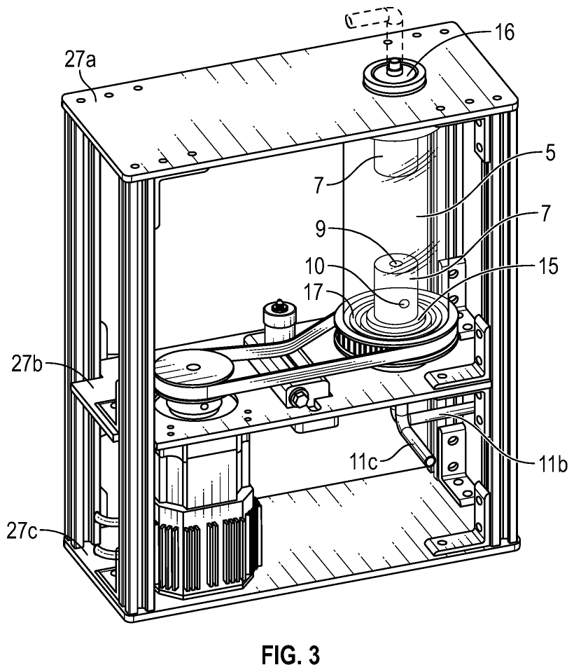

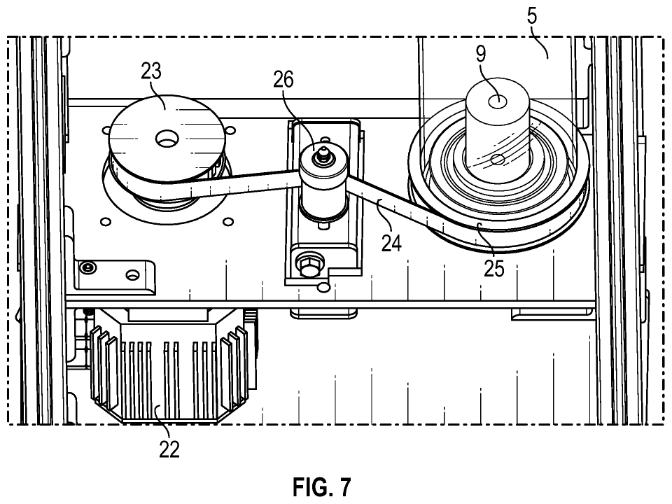

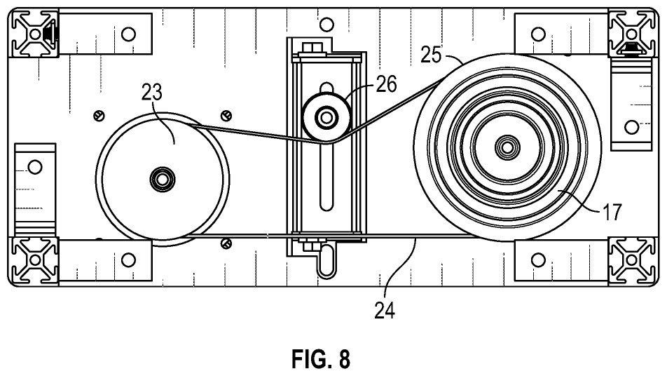

The continuous centrifugal isolation system (1) may further be configured to controllably rotate one or more individual or linked centrifugal columns (5). In one embodiment, a centrifugal column (5) may be controllably rotated by a motor (22) or other such appropriate device. Such rotation may be through a direct, or indirect coupling. In the preferred embodiment shown in FIGS. 3, and 7-8, a centrifugal column (5) may be controllably rotated by a belt drive having a motor (22) optionally mounted to the bottom plate (27c) of a scaffold (27) structure. This motor (22) may include or be coupled with to a rotational shaft (23) that may be responsive to a drive pully (23). A belt (24) may be coupled with the drive pully (23) and a centrifugal column (5), in this embodiment through a track (25) positioned on the outer surface of a lower cylinder support (18). A belt tensioner/idler pully (26), optionally mounted to a middle plate (27b) of a scaffold (27), may further be engaged with the belt (24) and configured adjust the rotational speed of the centrifugal column (5), or tension of the belt (24).

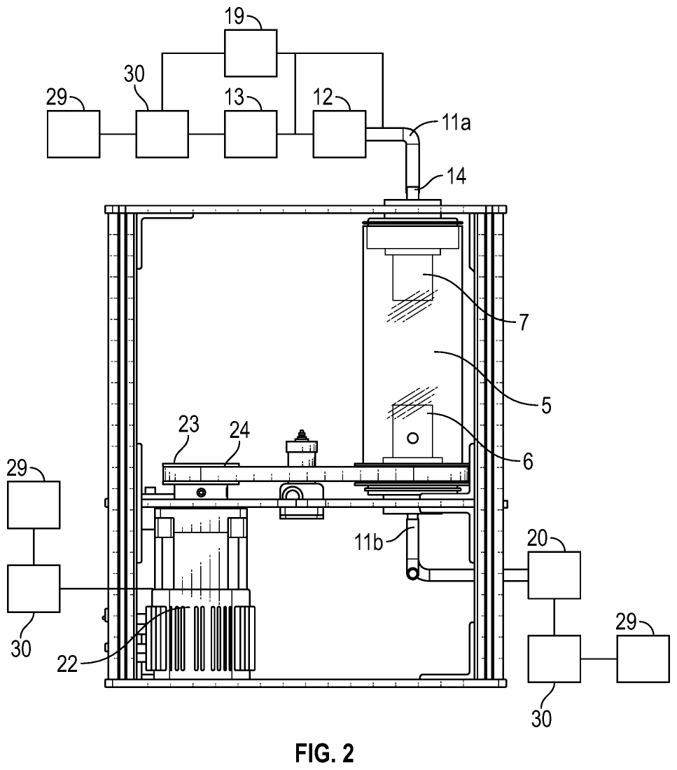

Generally referring to FIG. 2, the rotational rate of the centrifugal column (5) of the invention may be responsive to one or more sensors (30). The sensor (30) of the invention may generate a signal that may be transmitted to a digital device (29) having a processing system configured to effect one or more executable applications in response to the signal from one or more sensors (30) that may affect the rotation of the centrifugal column (5). This digital device may adjust the speed of the motor (22), the position of the belt tensioner/idler pully (26), or other gearing that may be responsive to the column.

As can be appreciated, some of the steps as herein described may be accomplished in some embodiments through any appropriate machine and/or device, such as a digital device (29) resulting in the transformation of, for example data, data processing, data transformation, external devices, operations, and the like. It should also be noted that in some instance's software and/or software solution may be utilized to carry out the objectives of the invention and may be defined as software stored on a magnetic or optical disk or other appropriate physical computer readable media including wireless devices and/or smart phones. In alternative embodiments the software and/or data structures can be associated in combination with a computer or processor that operates on the data structure or utilizes the software. Further embodiments may include transmitting and/or loading and/or updating of the software on a computer perhaps remotely over the internet or through any other appropriate transmission machine or device, or even the executing of the software on a computer resulting in the data and/or other physical transformations as herein described.

Certain embodiments of the inventive technology may utilize a machine and/or device which may include a digital devices (29), such as a general purpose computer, a computer that can perform an algorithm, computer readable medium, software, computer readable medium continuing specific programming, a computer network, a server and receiver network, transmission elements, wireless devices and/or smart phones, internet transmission and receiving element; cloud-based storage and transmission systems, software updateable elements; computer routines and/or subroutines, computer readable memory, data storage elements, random access memory elements, and/or computer interface displays that may represent the data in a physically perceivable transformation such as visually displaying said processed data. In addition, as can be naturally appreciated, any of the steps as herein described may be accomplished in some embodiments through a variety of hardware applications including a keyboard, mouse, computer graphical interface, voice activation or input, server, receiver and any other appropriate hardware device known by those of ordinary skill in the art.

A “processor,” “processor system,” or “processing system” includes any suitable hardware and/or software system, mechanism or component that processes data, signals or other information. A processor can include a system with a general-purpose central processing unit, multiple processing units, dedicated circuitry for achieving functionality, or other systems. Processing need not be limited to a geographic location or have temporal limitations. For example, a processor can perform its functions in “real time,” “offline,” in a “batch mode,” etc. Portions of processing can be performed at different times and at different locations, by different (or the same) processing systems. A computer may be any processor in communication with a memory. The memory may be any suitable processor-readable storage medium, such as random-access memory (RAM), read-only memory (ROM), magnetic or optical disk, or other tangible media suitable for storing instructions for execution by the processor.

Particular embodiments may be implemented by using a programmed digital device (30), such as a general purpose digital computer, by using application specific integrated circuits, programmable logic devices, field programmable gate arrays, optical, chemical, biological, quantum or nanoengineered systems, components and mechanisms may be used. In general, the functions of particular embodiments can be achieved by any means as is known in the art. Distributed, networked systems, components, and/or circuits can be used. Communication, or transfer, of data may be wired, wireless, or by any other means.

It will also be appreciated that one or more of the elements depicted in the drawings/figures can also be implemented in a more separated or integrated manner, or even removed or rendered as inoperable in certain cases, as is useful in accordance with a particular application. It is also within the spirit and scope to implement a program or code that can be stored in a machine-readable medium to permit a computer to perform any of the methods described above.

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise. As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise.

The foregoing description is merely illustrative in nature and is not intended to limit the embodiments of the subject matter or the application and uses of such embodiments. Furthermore, there is no intention to be bound by any expressed or implied theory presented in the technical field, background, or the detailed description. As used herein, the word “exemplary”, “embodiment” or “preferred embodiment” means “serving as an example, instance, or illustration.” Any implementation described herein as exemplary is not necessarily to be construed as preferred or advantageous over other implementations, and the exemplary embodiments described herein are not intended to limit the scope or applicability of the subject matter in any way.

For the sake of brevity, conventional techniques related to computer programming, computer networking, and other functional aspects of the systems (and the individual operating components of the systems) may not be described in detail herein. In addition, those skilled in the art will appreciate that embodiments may be practiced in conjunction with any number of system and/or network architectures, data transmission protocols, and device configurations, and that the system described herein is merely one suitable example. Furthermore, certain terminology may be used herein for the purpose of reference only, and thus is not intended to be limiting. For example, the terms “first”, “second” and other such numerical terms do not imply a sequence or order unless clearly indicated by the context.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application. Accordingly, details of the exemplary embodiments or other limitations described above should not be read into the claims absent a clear intention to the contrary.

As used herein, the terms “Microbubbles” and “bubbles” are used interchangeably and to refer to a gas core surrounded by a lipid membrane, which can be either a monolayer or a bilayer and wherein the lipid membrane can contain one or more lipids and one or more stabilizing agents. A microbubble may also mean a liposome and/or a micelle. In some embodiments, the microbubbles comprise one or more lipids. The term lipids includes agents exhibiting amphipathic characteristics causing it to spontaneously adopt an organized structure in water wherein the hydrophobic portion of the molecule is sequestered away from the aqueous phase. As described below, a microbubble may also contain target ligands, or other therapeutic agents, and/or other functional molecules, or may further include one or more gasses for example as provided in PCT/US2020/053627, incorporated herein it its entirety, including specifically compositions and methods of generating conjugated and/or cloaked microbubbles.

As used herein, a “dilute buoyant particle” means a particle that has a greater buoyancy that the suspension or solution material surrounding it. A dilute buoyant particle may include a microbubble, liposome, or micelle.

In some embodiments, the microbubble has a diameter size range that is about 3-5 μm. In some embodiments, the microbubble has a diameter size range that is about 1-5 μm. In some embodiments, the microbubble has a diameter size range that is about 4-5 μm. In some embodiments, the microbubble has a diameter size of about 4.5 μm. In another embodiment, the microbubble has a diameter size of about 4 μm or about 5 μm. In one embodiment, the microbubble has a diameter size of greater than 5 μm. In one embodiment, the microbubble has a diameter size of less than 1 μm.

As used herein, the general term biological marker (“receptors” “biomarker” or “marker” “moieties”) is a characteristic that is objectively measured and evaluated as an indicator of normal biologic processes, pathogenic processes, or pharmacological responses to therapeutic interventions, consistent with NIH Biomarker Definitions Working Group (1998). Markers can also include patterns or ensembles of characteristics indicative of particular biological processes. The biomarker measurement can increase or decrease to indicate a particular biological event or process. In addition, if the biomarker measurement typically changes in the absence of a particular biological process, a constant measurement can indicate occurrence of that process.

A target molecules or markers, and their corresponding interaction with a cloaked microbubble conjugated with a ligand, may be used for diagnostic and prognostic purposes, as well as for therapeutic, drug screening and patient stratification purposes (e.g., to group patients into a number of “subsets” for evaluation), as well as other purposes described herein.

The present invention includes all compositions and methods relying on correlations between the reported markers, cloaked microbubbles, and the therapeutic effect of cancer cells. Such methods include methods for determining whether a cancer patient or tumor is predicted to respond to administration of a therapy, as well as methods for assessing the efficacy of a therapy. Additional methods may include determining whether a cancer patient or tumor is predicted to respond to administration of a therapy. Further included are methods for improving the efficacy of a therapy, such as a cancer therapy, by administering to a subject a therapeutically effective amount of cloaked microbubble having one or more conjugated ligands that binds to, alters the activity of a biomarker, such as an angiogenesis markers such as integrin αVβ3, of VEGFR-2. In this context, the term “effective” is to be understood broadly to include reducing or alleviating the signs or symptoms of a disease condition, improving the clinical course of a disease condition, enhancing killing of cancerous cells, or reducing any other objective or subjective indicia of a disease condition, including indications of responsiveness to a treatment or non-responsiveness to a treatment, such as chemotherapy or radiation treatment. Different therapeutic microbubbles, doses and delivery routes can be evaluated by performing the method using different administration conditions.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “including” is used herein to mean, and is used interchangeably with, the phrase “including but not limited to.” The term “or” is used herein to mean, and is used interchangeably with, the term “and/or,” unless context clearly indicates otherwise.

1. A continuous centrifugal isolation system comprising:
a centrifugal column that holds a continuous flow of dilute buoyant particles, wherein said dilute buoyant particles comprise microbubbles;
a stationary input cylinder having at least one input port that inputs a dilute microbubble suspension into said centrifugal column;
wherein the rotational movement of said centrifugal column generates a centrifugal force causing the microbubbles disposed of therein to migrate to the center of the column and the lipid solution to migrate outward away from the center of the column;
a stationary output cylinder having at least one extraction port that extracts a quantity of concentrated microbubble suspension from said centrifugal column; and
at least one drain port on said stationary input cylinder that drains a quantity of excess lipid solution from said centrifugal column.

2. The system of claim 1, wherein said extraction port and said input port are configured in an opposing orientation so as to generate a fluid flow along the center of the column.

3. The system of claim 1, further comprising an extraction pump that generates a fluid flow along the center of the column.

4. The system of claim 1, further comprising an input pump that generates a fluid flow along the center of the column.

5. The system of claim 4, further comprising a reservoir, in fluid communication with said centrifugal column through an extraction pipe that receives said quantity of concentrated microbubble suspension from said centrifugal column.

6. The system of claim 4, further comprising an input pipe that conveys said quantity of dilute microbubble suspension to said centrifugal column.

7. The system of claim 4, further comprising a drain pipe that conveys said excess lipid suspension from said centrifugal column.

8. The system of claim 1, further comprising one or more annular bearings rotationally supporting said centrifugal column.

9. The system of claim 8, further comprising one or more cylinder supports coupling said centrifugal column and said annular bearing, and forming a water-tight seal thereof.

10. The system of claim 1, further comprising an input support coupled with said stationary input cylinder.

11. The system of claim 1, further comprising an output support coupled with said stationary output cylinder.

12. The system of claim 1, wherein said at least one drain port comprises one or more drain ports positioned laterally along on said stationary input cylinder.

13. The system of claim 1, and further comprising a drain pump that drains a quantity of the excess lipid solution from said centrifugal column.

14. The system of claim 1, further comprising a belt drive that rotates said centrifugal column having:
a motor;
a rotational shaft responsive to at least one drive pully;
a belt secured with said drive pully and a track responsive to said centrifugal column; and
a belt tensioner/idler pully.

15-16. (canceled)

17. The system of claim 1, wherein said continuous centrifugal isolation system comprises a plurality of continuous centrifugal isolation systems configured to operate independently or synchronously.

18. (canceled)

19. The system of claim 1, wherein said dilute microbubble suspension comprises a concentration of microbubbles of: 10⁸ MB/mL, or less than 10⁸ MB/mL.

20. The system of claim 1, wherein said dilute microbubble suspension comprises 5% encapsulated oxygen by volume.

21. The system of claim 1, wherein said concentrated microbubble suspension comprises 70% encapsulated oxygen by volume.

22. The system of claim 1, wherein said concentrated microbubble suspension comprises a concentration of microbubbles of: 10⁹ MB/mL, or greater than 10⁹ MB/mL.

23. The system of claim 1, or any above claim, wherein said centrifugal column exerts between 10-300 or more relative centrifugal force (RCF)/Gs during rotation.

24-58. (canceled)
